(19) United States
(12) Patent Application Publication
(10) Pub. No.: US 2021/0022597 A1
(43) Pub. Date: Jan. 28, 2021

(54) Probe Device

(71) Applicant: Kiran K. Bhat, Brooklyn, NY (US)

(72) Inventor: Kiran K. Bhat, Brooklyn, NY (US)

(21) Appl. No.: 17/063,687

(22) Filed: Oct. 5, 2020

Related U.S. Application Data
(63) Continuation-in-part of application No. 15/427,008, filed on Feb. 7, 2017.
(60) Provisional application No. 62/292,435, filed on Feb. 8, 2016.

(51) Int. Cl. A61B 1/31; G01R 1/067

(52) U.S. Cl. CPC A61B 1/31; A61B 2018/00964; G01R 1/06788

(57) ABSTRACT

A probe device designed to view, illuminate, and operate on the internal surface of an organ, configured to provide stabilization of the device with respect to the patient through a plug portion, manipulation of the probe through a skirt control system, which can be modified for the ergonomic preferences of the user, and permits discrete control over probe segments and segment cells through manipulation of the pleats which form the skirt.

### PRIORITY CLAIM

This application claims the benefit of and priority to U.S. Provisional Application Ser. No. 62/292,435, filed Feb. 8, 2016, and U.S. Non-Provisional application Ser. No. 15/427,008, filed Feb. 7, 2017, of which it is a CIP. The above referenced applications are incorporated in their entirety as if restated in full.

### BACKGROUND

There is a great need for medical probes capable of controlled entry and exploration of a patient's body, particularly in treating hemorrhoids. Generally, the probes are not flexible, or if they are flexible, are difficult to use based on inadequate controls. For example, these flexible probes are often only flexible in a single direction, requiring the user to rotate the device itself in order to rotate the direction of the probe. Also, the controls of the probe are usually fixedly integrated into the handles and manufactured in uniform sizes, thereby denying users the opportunity to manage a set of controls that is ergonomically preferable for them.

The probes tend to be unstable, increasing the difficulty in taking accurate diagnostic or surgical actions. Users of anal probes, specifically, encounter discomfort in the use of their devices because the use of their devices necessitate the expansion of and communication through the anus, which may express undesirable gasses and other fluid flow.

What is needed is a flexible probe configured to be operated easily by a user, adapt to the user's ergonomic preferences, affording the user a position of stability with which to operate the device, and provide some measure of protection against the environment created by the anus.

### SUMMARY

The probe device is designed to view, illuminate, and operate on the internal surface of a patient's organ. The device features a skirt control system which may be modified by the user to provide ergonomic conditions for the manipulation of a probe. The probe may be stabilized by a plug portion, which is connected to the skirt and provides a guide through which the probe may pass into the anus. The skirt features a set of pleats which are each coupled to segments of the probe, enabling the user to control the magnetic relationship between the segments, and consequently the curvature of the probe, by manipulating the pleats. Each segment is formed by a set of cells, and the magnetic relationship between cells in adjacent segments may be individually controlled by a pleat's “control positions”, which are orientations of the pleat with respect to a set of x, y, and z-axes.

### BRIEF DESCRIPTION OF THE DRAWINGS

FIG. 1 shows an exemplary probe device.

FIG. 2 shows a probe segment, which is a horizontal arrangement of cells.

FIG. 3 shows the probe, with the probe segments, in a straight configuration.

FIG. 4 shows the probe, with the probe segments in a curved configuration.

FIG. 5 shows a column, which is a vertical arrangement of cells, in a magnetized condition.

FIG. 6 shows the column in a demagnetized or repulsed condition.

FIG. 7 shows the skirt with the pleats extended.

FIG. 8 shows a pleat with an x, y, and z-axes projected to demonstrate the manipulation of the pleat toward various control positions.

FIG. 9 shows a method of using the probe device and an operation thereof.

### DETAILED DESCRIPTION

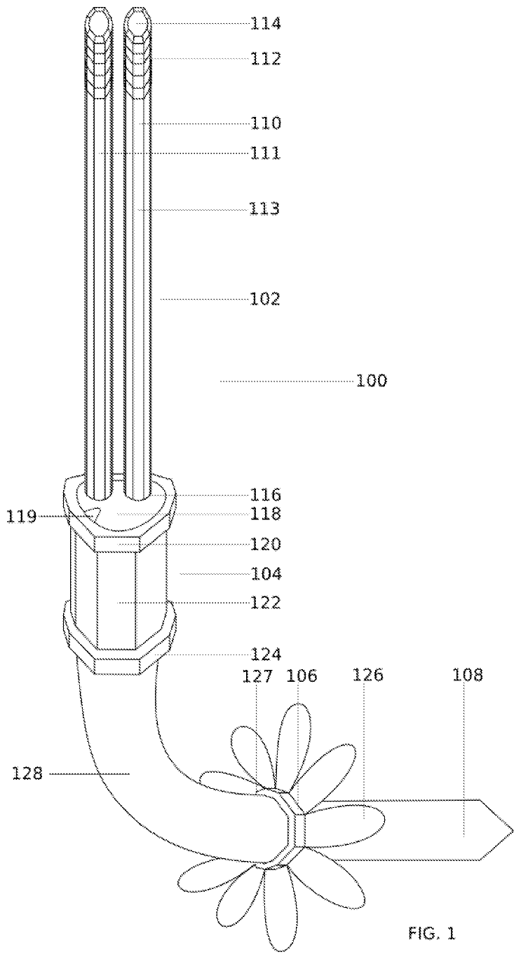

In a preferred embodiment, as shown in FIG. 1, the probe device 100 features a skirt 106, a handle 108, a plug portion 104, and a scope and probe portion 102, which should include both a light-emitting probe 110, and a camera scope 111.

The probe is a light emitting device that operates via a single or a series of lenses or optical fiber configured to amplify or focus and direct light, including infrared light, or lasers. The body of the probe is substantially cylindrical while in its straight configuration, although as will be described later, it can be manipulated to exhibit curvature. The probe may comprise a fixed portion 113 which cannot be manipulated by the skirt, as will be discussed below, and a portion which can be rigid or flexible, 112, depending on actions taken by the user. The probe has a top 114 from which the light emanates out of the probe.

The plug portion 104 is shaped somewhat like a grommet and configured to be inserted into a patient's anus. The plug portion may comprise a primary ring body 122, and a first and second ring collars 120 1124. The first ring collar has a larger outer diameter than the primary ring body so that when the primary ring body is pushed through the anus, particularly past the interior sphincter, the anus and interior sphincter will tighten around the primary ring body, adapt to the smaller diameter of the primary ring body, and thereby exert a pressure against the first ring collar if an accidental force exerts the plug portion back out of the anus. In this state, a greater force, such as an intentional force exerted by the user of the device, would be required to extract the plug portion from the anus.

The plug portion includes a membrane 118 which spans a cross section of the plug portion and which is designed to permit the passage of the probe and the scope through designated probe and scope openings 116 in the membrane. Thus, the membrane acts as a dual-guide, serving to maintain the position of the probe and scope with respect to the skirt. In a preferred variation, the membrane is capable of rotation independent of the primary ring, such that the membrane (including the respective positions of the scope and probe) can freely rotate even if the plug portion is rotationally stabilized by, for example, by being inserted and grasped firmly by the anus. The rotational independence is provided by providing a matching cylindrical cross section for the membrane outer diameter and the plug portion inner diameter. This independent motion may be “locked” via a latch fixing the membrane to the plug portion, or the alignment of interlocking teeth/gaps 119 at a designated “clock position”. Alternatively, the membrane and plug portion may be permanently integrated.

The plug portion and membrane should be at least one inch in length in order to better secure stability of the probe device as well as to maintain the probe's axial orientation.

The skirt 106 is essentially a controller for manipulating the curvature of the probe. It comprises a set of pleats 126 which join via ball-and-sockets or hinged at a waist 127, is disposed on the handle 108. The skirt may be directly connected to the plug portion, or attached via an intermediary adapter 128. The intermediary adapter share the same axis as the plug portion or may have curved or bent shape in order to provide the user, whose hands operate the skirt, a divergent trajectory from the anus.

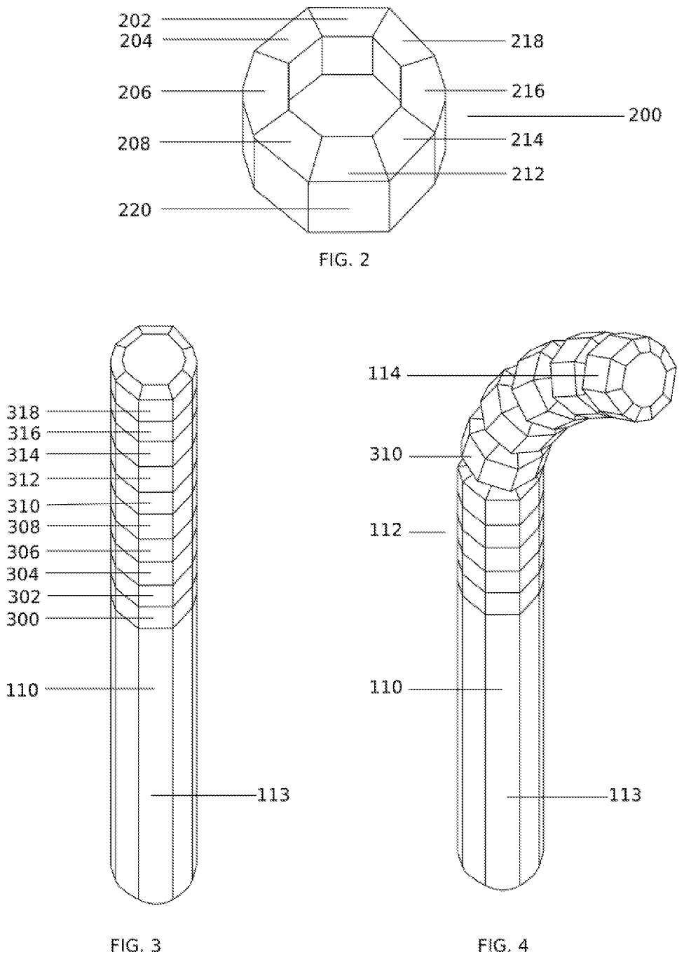

As shown in FIGS. 2-4, the probe body comprises a stack of row segments 200, with one segment 318 stacked on top of another segment 316, and so on 314 312 310 308 306 304 302 300. Each row segment is formed of a set of 4-8 cells 220, with the configuration of cells in one segment similar or identical to the configuration of cells above and/or below it, such that a series of cells are stacked on top of each other in a column, and 4-8 columns form the vertical span of the probe body. The columns may be designated by cardinal and/or intercardinal directions, such as north 202, north-west 204, west 206, south-west 208, south 212, south-east 214, east 216, and north-east 218. FIG. 3 shows the probe in a straight configuration, whereas FIG. 4 shows the probe curved after the 5^{th} segment 310, resulting in the tip 114 curved into a “j” shape, oriented 90 degree angle from the probe body. If additional segments were also “disengaged” like the first to fifth segments, the tip might curve into a “u” shape and thus oriented 180 degrees from the probe body.

The probe may obtain its light-emitting power from a bulb housing, which may be disposed at the base of the probe. The bulb housing may be configured to enclose a radiation emitting unit. This radiation emitting unit in turn may be electrically connected to the control assembly.

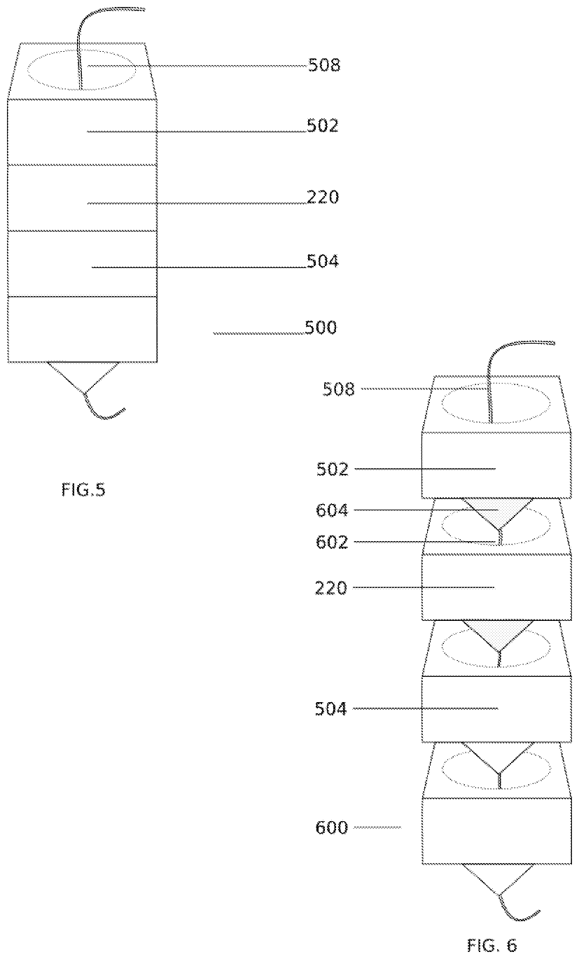

As shown in FIGS. 5-6, a column 500 of cells may be tightly stacked such that a first segment 220 is flush with one above if 502 as well as one below it 504. This tight stacking arrangement, which results in the probe body as a while being straight, is the result of electromagnets or the like embedded within the cells. A series of wires 508 runs through each column of cells, supplying electrical power to the electromagnets. These wires relay through a processor, preferably embedded in a control assembly disposed within the handle, carrying commands actuated by manipulations of the skirt pleats. As will be discussed later, each pleat, identified in the parent application as a “petal”, controls a segment, and manipulating the pleat in different ways results in magnetizing or demagnetizing various columns of the segment. As shown in FIG. 6, all four cells in the column 600, which also each occupy a segment, are demagnetized, resulting in their separation. While the term “demagnetized” is used, it is within the scope of this invention for the magnets to not only cease attracting, but begin repulsing. This can similarly be controlled by supplying electricity to the electromagnets via the wires. As shown in FIG. 6, the wires run through the conical cavities 602 and conical protrusions 604. The conical protrusions and cavities fit hand-in-glove, and thereby keep two adjacent cells rigid when the cavity of one is flush against the protrusions of the other. The conical protrusion is designed to fit in a flush manner against the conical intrusion, so that when the two segments are fully magnetically attracted and the distance is zero, minimal flexibility and therefore movement is possible. As the two segments separate, first a little, then more, the two segments have increasing flexibility and therefore movement. The degree of flexibility of the segments, and therefore the degree of curvature, are effected by the distance between one segment and another. This distance, and therefore the quality of degree of flexibility, is effected by the conical protrusion and intrusion relationship between any two adjacent cells.

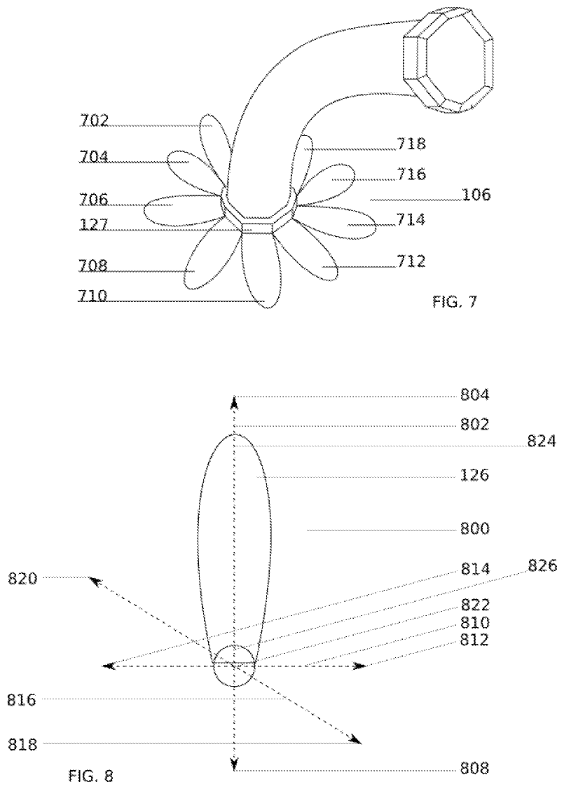

As shown in FIG. 7, the skirt comprises a waist 127 and a set of pleats, ideally equal in number to the number of flexible segments, so that each segment may have its own designated pleat to control it. Thus, a first pleat 702 controls the first segment 318, a second pleat 704 controls the second segment 316, a third pleat 706 controls the third segment 314, a fourth pleat 708 controls the fourth segment 312, a fifth pleat 710 controls the fifth segment 310, a sixth pleat 712 controls the sixth segment 308, a seventh pleat 714 controls the seventh segment 306, an eighth pleat 716 controls the eighth segment 304, a ninth pleat 718 controls the ninth segment 302, and a tenth pleat (not shown) controls the tenth segment 300. Each pleat can be manipulated to control the electromagnetic attractions between various cells in one segment with those of another segment. In one variation, a given pleat controls a set of more than one segment, affecting all the segments in a similar manner.

As shown in FIG. 8, the pleat 126, which may naturally extend along a z-axis 802 from a direction of the waist and handle 808 and away from the waist and handle 804. The pleat may attach to the waist via a ball-and socket 822 to allow for three dimensional motion, like certain levers. Indeed, the pleat may be shaped like a lever, although as described below, a pleat may be long, wide, but thin, like a leaf. While the base 826 of the pleat is stationary, the tip 824 of the pleat may be pushed along a y-axis 816, either in a “positive” direction 818 or a negative direction 820. Similarly, the tip may be pushed along an x-axis 810, either in a positive direction 812 or a negative direction 814. Movement of the tip purely in the positive direction of the y-axis changes the electromagnetism of the north cell, and movement of the tip purely in the negative direction of the y-axis changes the electromagnetism of the south cell. Similarly, movement of the tip purely in the positive direction of the x-axis changes the electromagnetism of the east cell, and movement of the tip purely in the negative direction of the x-axis changes the electromagnetism of the west cell. If the tip is moved in a direction between the positive direction of the x and y axes, this will change the electromagnetism of the north-east cell. A movement in a direction between the positive direction of the y axis and the negative direction of the x-axis changes the electromagnetism of the north-west cell. A movement in a direction between the negative direction of the y-axis and the negative direction of the x-axis changes the electromagnetism of the south-west cell. A movement in a direction between the negative direction of the y-axis and the positive direction of the x-axis changes the electromagnetism of the south-east cell. The orientation of the pleat may be determined by contact points between the ball and socket, such that a contact point on the pleat may engage with any of eight contact points in the socket, or vice versa. The electrical signal is relayed to a processor in the handle, which is then redirected via the wires previously discussed to each cell.

A “default position” for a given pleat, in which none of the electromagnetic elements are invoked, may be calibrated by the user. This default position, which may be set by the user and communicated to the processor through a “set” button or otherwise instructed programmatically, may be bent toward a positive or negative direction of the x and/or y axis. Deviations from this default position would then be recognized by the processor as instructions to invoke the electromagnetic elements and change the electromagnetism of the segment. By arranging the default positions of the pleats, the user can obtain an ergonomic configuration that works best for the size of the user's hand or based on other operational preferences.

The skirt also operates as a shield. The purpose of the shield is to block the projecting of gasses or liquids from the anus onto the hands of the user. When the pleats are fully extended, the outer diameter of the skirt may be between 5 and 7 inches. Each pleat may be shaped substantially trapezoidal or triangular in having an outer edge longer than the inner edge, which may be contracted to a point. The longer outer edge enables the pleats, when fully extended, to simultaneously fully block passage of air or other substances between the outer diameter and the waist by virtue of lacking gaps. In one variation, the sides of each pleat, which span from the outer edge to the waist, are each attached to the sides of adjacent pleats. These attachments may occur through the medium of a flexible material, such as vinyl.

The skirt operates as a controller, providing for both physical as well as mechanical manipulation of the probe device. The outer edges of the pleats provide a counterbalance by which the user can rotate the skirt around the center, which is an imaginary point positioned on the axis through which the skirt, and particularly the probe and scope, run. Physical rotation of the skirt may therefore result in rotation of the orientation of the membrane and thereby the scope and the probe.

Each pleat is electrically connected to an electromagnet embedded in a segment of the probe, and ideally, each pleat will be labelled with the segment to which it is coupled. The labelling may consist of a number, such as the “number” of the segment or a measurement distance from the waist, or the designated segment may be represented pictorially by distinguishing, through a color change or other graphical variation, between the segment that is designated and the segments that are not designated. Accordingly, each pleat may operate like a button, so that actuation of a given pleat actuates the electromagnet of its designated probe segment, thereby turning that electromagnet on or off. When an electromagnet is turned off, that segment is no longer magnetically attracted to its adjacent segment. In an ideal embodiment, turning off the electromagnet of a designated probe segment also turns off all electromagnets of subsequent probe segments. Once a probe segment is no longer magnetically attracted to adjacent segments, all segments thereafter become “limp”, and therefore flexible.

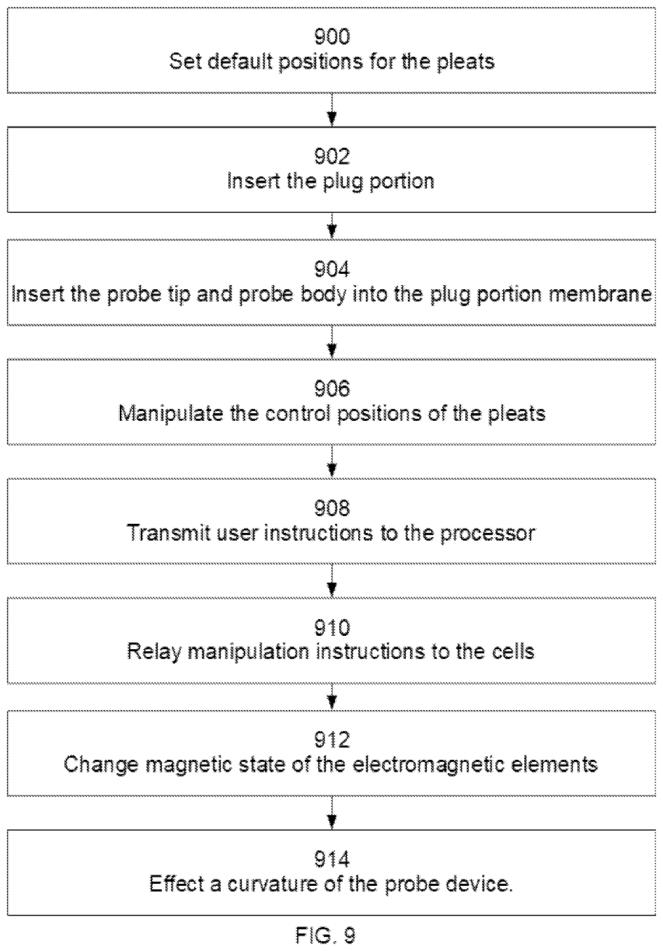

FIG. 9 shows a method of using the probe device and an operation thereof. First, the user may set the default positions for the pleats of the skirt 900. The plug portion may be inserted into a patient's anus 902, with the first collar embedded beyond the anus and the second collar before the anus, with the primary ring body clamped in place by the muscles of the anus. The probe tip, followed by the probe body, may be passed through the plug portion membrane and into the anus 904. While the step of setting the default positions for the pleats should certainly be taken prior to inserting the probe device, it is possible to insert the probe into the plug portion first, and thereafter insert the plug portion and the probe in simultaneously.

Thereafter, the user may manipulate the pleat control positions 906, which, through electrical contacts between the ball and socket jointure of the pleats to the waist, transmit the user instructions electrically to the processor 908. The processor may then relay these manipulation instructions along wires passing through the cells 910, which thereafter change the magnetic state of the electromagnetic elements 912, thereby effecting a curvature of the probe device 914.

1. A probe device for viewing and operating on an organ of a patient, comprising:
a. a skirt, a handle, a plug portion, a light-emitting probe, and a processor;
b. the handle configured to be held by a user to stabilize the probe device, the skirt attached to the handle and configured to be controlled by the user and permit the user to manipulate the probe, the plug portion configured to be inserted into the anus of the patient and stabilize the prove device with respect to the patient; the probe configured to emit light onto an interior portion of the organ, and the processor configured to process user commands received from the skirt and manipulate a curvature of the probe;
c. the probe comprising a flexible portion, the flexible portion configured to adapt a curvature and comprising sets of cells, each cell arranged horizontally with other cells in a segment and arranged vertically with other cells in a column;
i. each cell comprising an electromagnetic element, the electromagnetic element of any one cell configured to couple or decouple magnetically to electromagnetic elements in adjacent cells in the same column, with the electromagnetic elements connected electrically to the processor;
d. the plug portion comprising a primary ring body, a membrane, and first and second ring collars,
i. the first and second ring collars having diameters larger than a diameter of the primary ring body and disposed on opposite ends of the primary ring body;
ii. the primary ring body comprising an internal substantially cylindrical cavity configured to house the membrane;
iii. the membrane being disposed within the primary ring body cavity, comprising a probe opening, the probe opening configured to permit the probe to axially pass through the membrane;
iv. the membrane openings configured to exert a constant pressure on the probe in order to prevent the probe from accidentally sliding axially through the membrane;
v. the first ring collar configured to be pushed through the anus and prevent the plug portion from being accidentally removed from the anus, the second ring collar configured to be positioned outside the anus and impede the plug portion from accidentally fully entering the anus, and the primary ring body configured to maintain the anus in an open state, thereby permitting the probe and scope to pass through the membrane and into the anus;
e. the skirt comprising a set of pleats and a waist;
i. each pleat of the set of pleats attached to the waist;
ii. each pleat manipulatively coupled to a designated segment;
iii. each pleat having control positions, each control position controlling the electromagnetic element of a cell in the designated segment, with each control position communicated to the processor.

2. The probe device of claim 1, the probe also comprising a fixed portion and a tip, the tip attached to the flexible portion, and the flexible portion attached to the fixed portion, with the tip configured to emit light onto an interior surface of the organ of the patient.

3. The probe device of claim 1, each segment formed of 4-8 cells.

4. The probe device of claim 1, each cell comprising a protrusion and a cavity, the protrusion of any one cell configured to engage with the cavity of adjacent cells in the same column.

5. The probe device of claim 1, the membrane also comprising a scope opening, the scope opening configured to permit the scope to axially pass through the membrane.

6. The probe device of claim 1, the membrane configured to rotate independently from the primary ring body when a rotational force is applied.

7. The probe device of claim 6, the membrane having an interlocking tooth and the primary ring body having an interlocking gap, or the membrane having an interlocking gap and the primary ring body having an interlocking tooth, and the membrane configured to lock rotationally with respect to the primary ring body when the interlocking tooth aligns with the interlocking gap.

8. The probe device of claim 1, each pleat being labelled to identify the designated segment which it controls.

9. The probe device of claim 1, each pleat having 4-8 control positions.

10. The probe device of claim 1, each pleat of the set of pleats attached to the waist by a ball and socket.

11. The probe device of claim 8, each control position communicated to the processor through an actuation of contacts between electrical contacts on the ball and socket, which are then relayed to the processor.

12. The probe device of claim 1, the pleats configured to spread outward in a direction substantially orthogonal from the handle in order to provide a shield between the anus and the user.

13. A probe device for viewing and operating on an organ of a patient, comprising:
a. a skirt, a handle, a plug portion, a light-emitting probe, a camera scope, and a processor;
b. the handle configured to be held by a user to stabilize the probe device, the skirt attached to the handle and configured to be controlled by the user and permit the user to manipulate the probe, the plug portion configured to be inserted into the anus of the patient and stabilize the prove device with respect to the patient, the probe configured to emit light onto an interior portion of the organ, and the processor configured to process user commands received from the skirt and manipulate a curvature of the probe;
c. the probe comprising a fixed portion, a flexible portion, and a tip;
i. the tip attached to the flexible portion, and the flexible portion attached to the fixed portion, with the tip configured to emit light onto an interior surface of the organ of the patient;
ii. the flexible portion configured to adapt a curvature and comprising sets of cells, each cell arranged horizontally with other cells in a segment and arranged vertically with other cells in a column;
1. each segment formed of 4-8 cells;
2. each cell comprising an electromagnetic element, the electromagnetic element of any one cell configured to couple or decouple magnetically to electromagnetic elements in adjacent cells in the same column;
a. the electromagnetic elements connected electrically to the processor;
3. each cell comprising a protrusion and a cavity, the protrusion of any one cell configured to engage with the cavity of adjacent cells in the same column;
d. the plug portion comprising a primary ring body, a membrane, and first and second ring collars,
i. the first and second ring collars having diameters larger than a diameter of the primary ring body and disposed on opposite ends of the primary ring body;
ii. the primary ring body comprising an internal substantially cylindrical cavity configured to house the membrane;
iii. the membrane being disposed within the primary ring body cavity, comprising a probe opening and a scope opening, the probe opening and scope opening configured to permit the probe and scope, respectively, to axially pass through the membrane;
1. the membrane openings configured to exert a constant pressure on the probe and scope in order to prevent them from accidentally sliding axially through the membrane;
2. the membrane configured to rotate independently from the primary ring body when a rotational force is applied;
3. the membrane having an interlocking tooth and the primary ring body having an interlocking gap, or the membrane having an interlocking gap and the primary ring body having an interlocking tooth;
4. the membrane configured to lock rotationally with respect to the primary ring body when the interlocking tooth aligns with the interlocking gap;
iv. the first ring collar configured to be pushed through the anus and prevent the plug portion from being accidentally removed from the anus, the second ring collar configured to be positioned outside the anus and impede the plug portion from accidentally fully entering the anus, and the primary ring body configured to maintain the anus in an open state, thereby permitting the probe and scope to pass through the membrane and into the anus;
e. the skirt comprising a set of pleats and a waist;
i. each pleat of the set of pleats attached to the waist by a ball and socket;
ii. each pleat manipulatively coupled to a designated segment, with each pleat being labelled to identify the designated segment which it controls;
iii. each pleat having 4-8 control positions, each control position controlling the electromagnetic element of a cell in the designated segment, with each control position communicated to the processor through an actuation of contacts between electrical contacts on the ball and socket, which are then relayed to the processor;
iv. the pleats configured to spread outward in a direction substantially orthogonal from the handle in order to provide a shield between the anus and the user.

14. A probe device for viewing and operating on an organ of a patient, comprising:
a. a skirt, a plug portion, a light-emitting probe, and a processor;
b. the skirt configured to manipulate the probe, the plug portion configured to be inserted into the anus of the patient and stabilize the prove device with respect to the patient, the probe configured to emit light onto an interior portion of the organ, and the processor configured to process user commands received from the skirt and manipulate a curvature of the probe;
c. the probe comprising a flexible portion, the flexible portion configured to adapt a curvature and comprising sets of cells, each cell arranged horizontally with other cells in a segment and arranged vertically with other cells in a column;
i. each cell comprising an electromagnetic element, the electromagnetic element of any one cell configured to couple magnetically to electromagnetic elements in adjacent cells in the same column, with the electromagnetic elements connected electrically to the processor;
d. the plug portion comprising a primary ring body and a membrane,
i. the primary ring body comprising an internal substantially cylindrical cavity configured to house the membrane;
ii. the membrane being disposed within the primary ring body cavity, comprising a probe opening, the probe opening configured to permit the probe to axially pass through the membrane;
iii. the membrane openings configured to exert a constant pressure on the probe in order to prevent the probe from accidentally sliding axially through the membrane;
iv. the primary ring body configured to maintain the anus in an open state, thereby permitting the probe and scope to pass through the membrane and into the anus;
e. the skirt comprising a set of pleats and a waist;
i. each pleat of the set of pleats attached to the waist;
ii. each pleat manipulatively coupled to a designated segment;
iii. each pleat having control positions, each control position instructionally coupled to the electromagnetic element of a cell in the designated segment, with each control position communicated to the processor and then relayed to control the electromagnetic element of the cell in the designated segment.

15. The probe device of claim 14, also comprising a handle, the handle configured to be held by the user.

16. The probe device of claim 15, the pleats configured to spread outward in a direction substantially orthogonal from the handle in order to provide a shield between the anus and the user.

17. The probe device of claim 14, the plug portion also comprising first and second ring collars, the first and second ring collars having diameters larger than a diameter of the primary ring body and disposed on opposite ends of the primary ring body, the first ring collar configured to be pushed through the anus and prevent the plug portion from being accidentally removed from the anus, the second ring collar configured to be positioned outside the anus and impede the plug portion from accidentally fully entering the anus.

18. The probe device of claim 14, each pleat of the set of pleats attached to the waist by a ball and socket.

19. The probe device of claim 18, each control position communicated to the processor through an actuation of contacts between electrical contacts on the ball and socket, which are then relayed to the processor.

20. The probe device of claim 1, the membrane configured to rotate independently from the primary ring body when a rotational force is applied.
